(19) United States
(12) Patent Application Publication
(10) Pub. No.: US 2019/0236678 A1
(43) Pub. Date: Aug. 1, 2019

(54) SYSTEMS AND METHODS FOR RECOMMENDING ITEMS FOR PURCHASE

(71) Applicant: Walmart Apollo, LLC, Bentonville, AR (US)

(72) Inventors: Bruce W. Wilkinson, Rogers, AR (US); Brian G. McHale, Chadderton Oldham (GB); Todd D. Mattingly, Bentonville, AR (US); Kurt W.R. Bessel, Mexico, NY (US)

(21) Appl. No.: 16/255,098

(22) Filed: Jan. 23, 2019

Related U.S. Application Data
(60) Provisional application No. 62/622,611, filed on Jan. 26, 2018.

(51) Int. Cl. G06Q 30/06; G01N 33/00; G01N 33/02

(52) U.S. Cl. CPC G06Q 30/0631; G01N 33/0001; G01N 33/02; G01N 33/0063

(57) ABSTRACT

In some embodiments, apparatuses and methods are provided herein useful to recommend items. In some embodiments, there is provided a system for recommending items for purchase based on smell of food including a user interface product configured to trigger an odor sensor, prompt a user to provide an indication, receive a location coordinate, and provide a listing of identified chemicals, the indication, and the location coordinate; a main database configured to store a first set of associations and a second set of associations; and a main control circuit configured to determine a filter parameter, determine a filtered set of recipe components from the first set of associations, determine at least one recipe component from the filtered set of recipe components, determine a filtered set of recipes from the second set of associations, determine one or more recipes from the filtered set of recipes, and provide the one or more recipes.

### CROSS-REFERENCE TO RELATED APPLICATION

This application claims the benefit of U.S. Provisional Application No. 62/622,611 filed Jan. 26, 2018, which is incorporated herein by reference in its entirety.

### TECHNICAL FIELD

This invention relates generally to recommending items for purchase.

### BACKGROUND

Generally, when a customer is at a restaurant and smells an odor he/she may like, the customer may look around to see the various entrees the other customers may have ordered. The customer may point the server to the entrees that look appealing and ask for the name of the entrees. However, the entrees identified by the server may not correspond to the odor the customer likes.

### BRIEF DESCRIPTION OF THE DRAWINGS

Disclosed herein are embodiments of systems, apparatuses and methods pertaining to recommending items for purchase. This description includes drawings, wherein:

FIG. 1 illustrates a simplified block diagram of an exemplary system for recommending items for purchase based on smell of food in accordance with some embodiments;

FIG. 2 shows a flow diagram of an exemplary process of recommending items for purchase based on smell of food in accordance with some embodiments;

FIG. 3 shows a flow diagram of an exemplary process of recommending items for purchase based on smell of food in accordance with some embodiments;

FIG. 4 shows a flow diagram of an exemplary process of recommending items for purchase based on smell of food in accordance with some embodiments;

FIG. 5 shows a flow diagram of an exemplary process of recommending items for purchase based on smell of food in accordance with some embodiments;

FIG. 6 shows a flow diagram of an exemplary process of recommending items for purchase based on smell of food in accordance with some embodiments; and

FIG. 7 illustrates an exemplary system for use in implementing methods, techniques, devices, apparatuses, systems, servers, sources, and recommendation of items for purchase based on smell of food, in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to various embodiments, systems, apparatuses and methods are provided herein useful for recommending items for purchase based on one or more orders or smells. In some embodiments, a system recommending items for purchase based on smell of food includes a user interface product. By one approach, the user interface product may be operable on a portable user electronic device. In one configuration, the portable user electronic device may include an odor sensor. In one example, the odor sensor may detect chemicals associated with an odor. In another configuration, the portable user electronic device may include a transceiver and/or a portable user electronic device control circuit. In one example, the portable user electronic device control circuit may identify the chemicals detected by the odor sensor. Alternatively or in addition to, a user interface product may be cooperated with the portable user electronic device control circuit. In one example, the user interface product may trigger the odor sensor to initiate detection of the chemicals associated with the odor based on a first user input through the portable user electronic device. By one approach, the user interface product may prompt a user to provide an indication of a measure of like or a measure of dislike of the odor through a second user input via the portable user electronic device. By another approach, the user interface product may receive a location coordinate corresponding to a current location of the portable user electronic device. By yet another approach, the user interface product may provide a listing of the identified chemicals, the indication, and/or the location coordinate via the transceiver through an internet network. Alternatively or in addition to, the system may include a main database. In one configuration, the main database may be communicatively coupled to the portable user electronic device control circuit and/or the user interface product via the internet network. By one approach, the main database may store a first set of associations of a plurality of recipe components with a plurality of corresponding set of chemicals and a second set of associations of a plurality of recipes with a plurality of corresponding set of recipe components. In another configuration, the system may include a main control circuit. In one example, the main control circuit may communicatively couple to the main database, the portable user electronic device control circuit, and/or the user interface product via the internet network. By one approach, the main control circuit may receive the listing of the identified chemicals, the indication, and/or the location coordinate. By another approach, the main control circuit may, in response to the indication of the measure of like, determine a filter parameter associated with the location coordinate. By another approach, based on the filter parameter, the main control circuit may determine a filtered set of recipe components from the first set of associations of the plurality of recipe components stored in the main database. By another approach, the main control circuit may determine at least one recipe component from the filtered set of recipe components that has a chemical makeup including one or more of the chemicals identified in the listing of the identified chemicals. By another approach, the main control circuit may, based on the filter parameter, determine a filtered set of recipes from the second set of associations of the plurality of recipes stored in the main database. By yet another approach, the main control circuit may determine one or more recipes from the filtered set of recipes that includes the determined at least one recipe component. In one configuration, the main control circuit may provide the one or more recipes to the user interface product via the internet network.

In some embodiments, a method for recommending items for purchase based on one or more orders or smells includes triggering, by a user interface product operable on a portable user electronic device, an odor sensor cooperated with and/or part of the portable user electronic device to initiate detection of chemicals associated with an odor based on a first user input through the portable user electronic device. In one configuration, the method may include prompting, by the user interface product, a user to provide an indication of a measure of like or a measure of dislike of the odor through a second user input via the portable user electronic device. In another configuration, the method may include receiving, by the user interface product, a location coordinate corresponding to a current location of the portable user electronic device. In another configuration, the method may include providing, by the user interface product, a listing of identified chemicals, the indication, and/or the location coordinate via an internet network through a transceiver of the portable user electronic device. By one approach, the identified chemicals may be detected by the odor sensor and identified by a portable user electronic device control circuit of the portable user electronic device. In one configuration, the method may include receiving, by a main control circuit communicatively coupled to a main database, the portable user electronic device control circuit, and/or the user interface product via the internet network, the listing of the identified chemicals, the indication, and/or the location coordinate. In one example, the main database may store a first set of associations of a plurality of recipe components with a plurality of corresponding set of chemicals and a second set of associations of a plurality of recipes with a plurality of corresponding set of recipe components. In another configuration, the method may include determining a filter parameter associated with the location coordinate in response to the indication corresponding to the measure of like. In another configuration, the method may include, based on the filter parameter, determining a filtered set of recipe components from the first set of associations of the plurality of recipe components stored in the main database. In another configuration, the method may include determining, by the main control circuit, at least one recipe component from the filtered set of recipe components that has a chemical makeup including one or more of the chemicals identified in the listing of the identified chemicals. In another configuration, the method may include, based on the filter parameter, determining, by the main control circuit, a filtered set of recipes from the second set of associations of the plurality of recipes stored in the main database. In another configuration, the method may include determining, by the main control circuit, one or more recipes from the filtered set of recipes that includes the determined at least one recipe component. In yet another configuration, the method may include providing, by the main control circuit, the one or more recipes to the user interface product via the internet network.

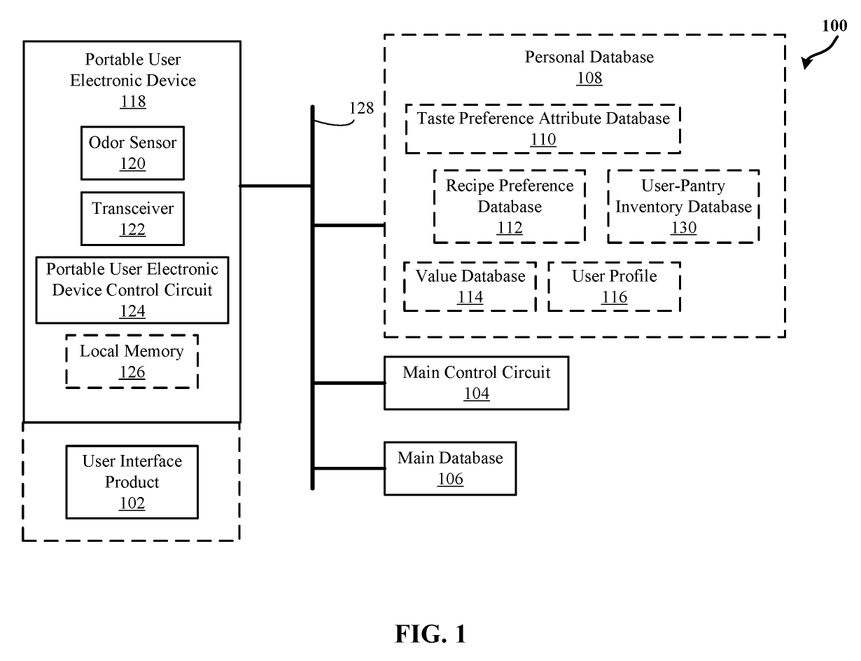

FIG. 1 illustrates a simplified block diagram of an exemplary system 100 that recommends items for purchase based on a smell, order or the like of food or other odor emitting element in accordance with some embodiments. The exemplary system 100 includes a user interface product 102. By one approach, the user interface product 102 may be operable on a portable user electronic device 118. By another approach, the user interface product 102 may be integrated with the portable user electronic device 118. In one configuration, the user interface product 102 may correspond to an electronic device configured to couple communicatively and/or physically, and operate in cooperation with the portable user electronic device 118. In another configuration, the user interface product 102 may correspond to a computer program product embodied on a computer readable storage medium, for example, a local memory 126 of the portable user electronic device 118. In one example, the computer readable storage medium may comprise a read only memory (ROM), an electrically erasable programmable read-only memory (EEPROM), a random-access memory (RAM), a hard disk drive, a secure digital card, a non-volatile memory device, a volatile memory device, and/or other memory devices and data storage devices configured to store electronic data and/or digital data. In one configuration, the system 100 may include the portable user electronic device 118. In one example, the portable user electronic device 118 may comprise a laptop, a smartphone, an iPad, a tablet, a smartwatch, and/or any electronic device that is easily carriable by a user from one place to another and/or carriable on the user's person. In another example, the portable user electronic device 118 includes a transceiver 122 and a portable user electronic device control circuit 124. Further, the portable user electronic device 118 includes and/or communicatively couples with an odor sensor 120 or other such chemical sensor system configured to process chemicals carried by air. For example, the odor sensor may be a portable odor meter from Kanomax USA, Inc., a Cyranose Electronic Nose sensor from Sensigent, nanocomposite sensors from Sensigent, other such sensor systems, or combination of two or more of such sensor systems.

In another configuration, the system 100 may include a personal database 108, a taste preference attribute database 110, a recipe preference database 112, a value database 114, a user-pantry inventory database 130, and/or a user profile 116. In one example, the personal database 108 may comprise the taste preference attribute database 110, the recipe preference database 112, the value database 114, the user-pantry inventory database 130, and/or the user profile 116. In another example, one or more of the personal database 108, the taste preference attribute database 110, the recipe preference database 112, the value database 114, the user-pantry inventory database 130, and/or the user profile 116 may be integrated with the portable user electronic device 118. In yet another example, some or all of one or more of the personal database 108, the taste preference attribute database 110, the recipe preference database 112, the value database 114, the user-pantry inventory database 130, and/or the user profile 116 may be accessible through one or more database systems and/or memory distributed over one or more internet networks 128. By one approach, the internet network 128 may include a local area network, a WiFi network, a cellular network, and/or any communication network used as a medium of communication between one electronic device and one or more electronic devices.

In another configuration, the system 100 may include a main control circuit 104. In one example, the main control circuit 104 may be remote from and communicatively coupled with the portable user electronic device 118 and/or the user interface product 102. In such an example, the main control circuit 104 may be operated on a computer server and/or a distributed computing system. In another example, the portable user electronic device 118 may include the main control circuit 104. In yet another configuration, the system 100 may include a main database 106. In one example, the main database 106 may be distinct from the personal database 108, the taste preference attribute database 110, the recipe preference database 112, the value database 114, and/or the user profile 116. In another example, the main database 106 may include part of or all of one or more of the personal database 108, the taste preference attribute database 110, the recipe preference database 112, the value database 114, and/or the user profile 116.

In yet another configuration, the main control circuit 104 may be communicatively coupled with one or more of the personal database 108, the taste preference attribute database 110, the recipe preference database 112, the value database 114, the user profile 116, the main database 106, the user interface product 102, and/or the portable user electronic device 118 via the internet network 128.

In some embodiments, the user interface product 102 may be operable on the portable user electronic device 118. By one approach, the user interface product 102 may trigger the odor sensor 120 to initiate detection of chemicals associated with an odor based on a first user input through the portable user electronic device 118. For example, a user may like an odor smelled while at a restaurant, passing by a restaurant, or at a place where an odor may be present and smellable by a user. In one scenario, the user may provide a first input that initiate the user interface product 102 to trigger the odor sensor 120 to start detecting the chemicals associated with the odor. In one example, the first input may be in a form of the user initiating the user interface product 102, pressing displayed option or button presented by the user interface product 102, and/or a physical button on the portable user electronic device 118 that is cooperated with the user interface product 102. In one configuration, the portable user electronic device control circuit 124 in cooperation with the odor sensor 120 may identify the chemicals of the odor detected by the odor sensor 120. By one approach, the user interface product 102, in cooperation with the portable user electronic device control circuit 124 or operating on its own, prompt a user to provide an indication of a measure of like or a measure of dislike of the odor through a second user input via the portable user electronic device 118. In one scenario, the indication may include a rating of an odor by a user. In one example, the rating may correspond to the user's degree of likeness of the odor and/or the user's degree of dis-likeness of the odor. In another example, the rating may correspond to the user's degree of emotional response elicited by the odor. For example, a user may rate the odor based on how the odor makes the user feel comfortable (e.g., the odor reminds the user of the user's childhood) and/or invigorated, to name a few. In another scenario, the measure of like or a measure of dislike may comprise a range of numbers indicative of a degree of likeness or dislikeness, a numerical number rating, a star number rating, or the like. In some instances, a graphical user interface is displayed on a display of the portable user device 118 that allows the user to specify a level of like or dislike. For example, the graphical user interface may allow a user to select a like option or a dislike option, and specify a corresponding level of like or dislike (e.g., enter a number between 1 and 10, move a graphically displayed dial, slide a graphically displayed bar, etc.), a dial may be displayed where an amount of a virtual turn in one direction indicates a level of like and an amount of virtual turn in another direction indicates a level of dislike, a graphical bar may be displayed with a slider that can be virtually moved by the user to indicate a level of like or dislike, or other such mechanism to indicate a level of like or dislike. In some instances, the display of this graphical interface prompts the user to provide an indication of a measure or level of like (e.g., like or dislike). Alternatively or in addition to, the user interface product 102 may receive a location coordinate corresponding to a current location of the portable user electronic device 118. In one example, the location coordinate may be received by the user interface product 102 when the odor sensor 120 is triggered to initiate detection of the chemicals associated with the odor. In another example, the location coordinate may be received by the user interface product 102 when the user provides the indication of the measure of like or the measure of dislike of the odor.

By one approach, the user interface product 102 may provide a listing of the identified chemicals, the indication, and/or the location coordinate to the main control circuit 104 via the transceiver 122 through the internet network 128. In one configuration, in response to the indication of the measure of like, the main control circuit 104 may determine a filter parameter associated with the location coordinate. Alternatively or in addition to, in response to the indication of the measure of dislike, the main control circuit 104 may perform one or more functions described herein to determine the odor and/or the chemicals disliked by the user and use the resulting determination to efficiently and/or quickly determine odors and/or chemicals that are subsequently indicated by the user as having a measure of like by filtering out those odors and/or chemicals disliked by the user from a list to be searched from. For example, the main control circuit 104 may store the resulting determination in one of the databases described herein and/or in the user profile 116 that is associated with a user. In such an example, a subsequent determination of an odor smelled by a user may be determined from a listing of odors and/or chemicals that does not include odors and/or chemicals previously indicated by the user as having a measure of dislike.

By another approach, the main control circuit 104 may, based on the filter parameter, determine a filtered set of recipe components from a first set of associations of a plurality of recipe components stored in the main database 106. In one example, the main database 106 may store the first set of associations of the plurality of recipe components with a plurality of corresponding set of chemicals and/or a second set of associations of a plurality of recipes with a plurality of corresponding set of recipe components. To illustrate, a first association of basil with its corresponding mass spectrum data, a second association of basil with its corresponding chemical components, and/or a third association of basil with one or more dishes having recipes that include basil (e.g., pesto sandwich, pesto sauce, etc.) may be stored in the main database 106. The foregoing is just an example illustration of data that may be stored and/or associated in the main database 106 and is not to be construed as limiting the kind, sort, variety, classes, categories, or sets of recipe components, corresponding set of chemicals (for example, mass spectrum data, mass-to-charge ratio, mass spectra, molecular mass, or the like), and/or recipes that may be stored and/or associated in the main database 106.

By another approach, the main control circuit 104 may determine one or more recipe components from a filtered set of recipe components that has a chemical makeup including one or more of chemicals identified in a listing of identified chemicals provided by the user interface product 102 and/or the portable user electronic device 118. Continuing the illustrative non-limiting example above, the user may be walking passed an Italian restaurant and smelled an odor of a dish. After triggering the odor sensor 120 to detect chemicals associated with the odor, the user interface product 102 may receive a location coordinate of the user via the portable user electronic device 118. In one example, the user interface product 102 and/or the main control circuit 104 may determine that the location coordinate is within a threshold distance from an Italian restaurant based, in part, on stored satellite mappings of at least one of commercial buildings and businesses, landmarks, and/or major point of interests accessible by the user interface product 102 and/or the main control circuit 104. In an example, the main control circuit 104 may correlate the location coordinate with the stored satellite mappings and determine that the location coordinate associated with the portable user electronic device 118 corresponds to or within a threshold distance from an Italian restaurant. As such, in this example, the main control circuit 104 may determine a filter parameter that is associated with an Italian restaurant. In this non-limiting example, the filter parameter may include Italian ingredients, Italian recipes, Italian cooking, Italian food, among other filter parameters that maybe associated with an Italian restaurant to indicate that particular recipe components that the user interface product 102 and/or the main control circuit 104 may be directed to determine those recipe components that are used and/or included in preparing an Italian dish. By one approach, a filter parameter may include one or more descriptive words, indexes, triggers, tags, or the like associated with a location coordinate of the portable user electronic device 118 at a particular time determined when a user trigger the user interface product 102 to detect an odor.

Continuing the non-limiting, illustrative example above, the user may have smelled the pesto sauce on a dish served by the restaurant. In this example, the listing of identified chemicals provided by the user interface product 102 may include chemical components corresponding to basil. By one approach, the main control circuit 104 may use the filter parameter (e.g., the Italian ingredients) associated with the Italian restaurant to narrow down the set of chemicals to compare the listing of identified chemicals associated with the odor smelled by the user. As such, the filtered set of recipe components determined by the main control circuit 104 may be associated with only chemicals that are associated with the Italian ingredients. In one configuration, the main control circuit 104 may determine one or more recipe components of the odor from a filtered set of recipe components that has the chemical makeup corresponding to ingredients found in Italian dishes, for example. Thus, the main control circuit 104 may efficiently and/or swiftly determine that one of the recipe components associated with the odor that spark the user's interest is basil.

Alternatively or in addition to, the main control circuit 104 may, based on a filter parameter associated with a location coordinate, determine a filtered set of recipes from the second set of associations of the plurality of recipes stored in the main database 106. Thus, the main control circuit 104 may efficiently and/or swiftly determine the filtered set of recipes using the filter parameter. Continuing the illustrative non-limiting example above, the main control circuit 104 may use the filter parameter (e.g., the Italian ingredients) associated with the Italian restaurant to filter out those recipes that are not associated with the Italian ingredients. By narrowing to a set of recipes (e.g., the filtered set of recipes) to search from, where each recipe of the set may have in common at least one filter parameter, the main control circuit 104 may, for example, efficiently and/or swiftly determine one or more recipes that include basil as one of ingredients.

In some embodiments, the main control circuit 104 may provide one or more recipes to the user interface product 102 via the internet network 128. Continuing the non-limiting, illustrative example above, the user interface product 102 may display on a display device of the portable user electronic device 118 a listing of recipes that include basil as one of the ingredients. By one approach, the user interface product 102 may provide a first message to the user of the portable user electronic device 118 prompting the user to select one of the recipes shown on the display device, for example. Alternatively or in addition to, the user interface product 102 may provide a second message to the user prompting the user whether the user would like to submit an order for one or more recipe components of the selected recipe. For example, the user may select a recipe for a pork belly sandwich topped with pesto sauce. The user may then submit an order to a retailer for a delivery of a package of pork belly meat and/or a package of basil via the user interface product 102.

In one configuration, the value database 114 may store a plurality of recipe attributes and corresponding number of times each attribute of the plurality of recipe attributes is associated with a selected recipe. For example, the user interface product 102 may provide a third message to the user prompting the user to select an attribute and/or a corresponding attribute rating value to be associated with the selected recipe. By one approach, at a given period of time, a number of times a recipe attribute is associated with a selected recipe is tallied by the user interface product 102 and/or the main control circuit 104. Alternatively or in addition to, the user interface product 102 may prompt the user to provide an attribute rating value to each of a plurality of recipe attributes relative to each of the recipes provided by the main control circuit 104 and selectable by the user. For one example, based on the recipes provided by the main control circuit 104, the user interface product 102 may show a listing of a number of recipes and each recipe on the listing has corresponding attributes to be rated by the user. By one approach, the user interface product 102 may prompt the user to provide an attribute rating value to each of the corresponding attributes of each recipe on the listing. In one example, the attributes may include simple foods, easy to cook, fresh ingredients, vegan, complicated, and/or quick preparation, among other attributes that may be associated with various recipes. In an illustrative non-limiting example, the user may assign a rating value of 8 out of 10 to a recipe attribute that corresponds to quick preparation for a basic pesto sauce recipe. Alternatively or in addition to, the user interface product 102 may tally, for example periodically and/or when triggered to tally, the attribute rating values associated with each recipe attributes corresponding to one or more recipes in the value database 114.

Continuing the illustrative non-limiting example above, the user may have triggered a number of times the user interface product 102 to determine an odor corresponding to basil and selected the same recipe of pesto sauce. In one configuration, the user interface product 102 and/or the main control circuit 104 may sum the number of times a particular attribute is associated with the pesto sauce recipe, average the rating values associated with the particular attribute, initiate storing and/or associating of the sum, and/or the average with the particular attribute in the value database 114. By one approach, those attributes with an average rating value of 8 out of 10 and/or 80%, for example, may correspond to attributes of recipes that the user may find very desirable. Alternatively or in addition to, the value database 114 may store the plurality of recipe attributes with associated attribute rating values provided by the user over a time period. In one example, the associated attribute rating values may be updated each time the user provides recipe attribute ratings to each of the recipes provided by the main control circuit 104.

In another configuration, the user interface product 102 may receive one or more recipe components via the internet network 128 from the main control circuit 104. Alternatively or in addition to, the user interface product 102 may provide a message to a user of the portable user electronic device 118 prompting the user to provide a taste preference rating value to the one or more recipe components. In response to receiving the taste preference rating value, the user interface product 102 may associate, in cooperation with the portable user electronic device control circuit 124, the taste preference rating value and/or a taste preference with one or more recipe components in the taste preference attribute database 110. Continuing the illustrative non-limiting example above, the user may associate, for example, a taste preference rating value of 4 out of 5 possible taste preference rating values with basil. Alternatively or in addition to, the user may associate a taste attribute of refreshing with basil along with the taste preference rating value in the taste preference attribute database 110. In one example, the taste attribute may include refreshing, spicy, salty, and/or sweet, among other various descriptions attributable to a taste of an ingredient. By one approach, the taste preference attribute database 110 may store a plurality of determined recipe components with associated plurality of taste preference rating values provided by the user over a time period. In one example, the plurality of determined recipe components may be based on chemicals detected by the odor sensor 120 over the time period.

In another configuration, the recipe preference database 112 may store a listing of recipes based on chemicals detected by the odor sensor 120 over a time period and corresponding recipe preference rating value provided by the user. For example, the user interface product 102 may provide a message to a user of the portable user electronic device 118 prompting the user to provide a recipe preference rating value to one or more recipes received from the main control circuit 104 and/or associate the recipe preference rating value and/or the one or more recipes with one or more recipe components detected by the odor sensor 120. By one approach, in associating a recipe preference rating value and/or a recipe with one or more recipe components, the user may indicate to the main control circuit 104 and/or the user interface product 102 that the recipe is given a particular rating by the user based on particular recipe components of the recipe. Alternatively or in addition to, in providing a number to associate with the recipe preference rating value, the user may indicate a degree of desirability and/or appeal, from a user's perspective, the corresponding recipe received from the main control circuit 104. In such an example, the user interface product 102 may associate, in cooperation with the portable user electronic device control circuit 124, a recipe preference rating value with a recipe in the recipe preference database 112.

In some embodiments, the main control circuit 104 may select a user-centric-based recipe from one or more recipes determined from a filtered set of recipes based on recipe preference rating value associated with each recipe in the recipe preference database 112, attribute rating values of the value database 114, and a plurality of taste preference rating values of the taste preference attribute database 110. For example, over a period of time, the main control circuit 104 may have collected, associated, and/or initiated storage of rating values in one or more of the database described herein. By one approach, the stored rating values may be associated with the user profile 116 of the user. Alternatively or in addition to, based on the stored rating values and subsequent to determining one or more recipe components of an odor smelled by the user, the main control circuit 104 may select a user-centric-based recipe to provide to the user interface product 102. In a non-limiting illustrative example, the determined recipe components of an odor may include turmeric, tomato, onions, and/or ginger. In one configuration, in processing the stored rating values, the main control circuit 104 may determine that among the recipes associated with tumeric, tomato, onions, and/or ginger, the user has the highest rating value for easy to cook and tend to frequently trigger the user interface product 102 to determine an odor corresponding to turmeric. As such, the main control circuit 104 may select a recipe that is associated with easy to cook and includes turmeric. By one approach, the main control circuit 104 may accessed the main database 106 and/or other databases described herein and may determine that a chicken curry recipe is associated with having turmeric as an ingredient and an attribute corresponding to easy to cook. In response, the main control circuit 104 may provide a recipe for chicken curry to the user through the user interface product 102. Thus, the main control circuit 104 may provide the user-centric-based recipe to the user interface product 102 via the internet network 128.

Continuing the non-limiting illustrative example above, the user interface product 102 may prompt the user whether the user would like to submit an order for one or more of the recipe components (e.g., turmeric and chicken) of the chicken curry recipe. As such, alternatively or in addition to, the user interface product 102 may provide a first message to a user of the portable user electronic device 118 prompting the user whether the user would like to submit an order for one or more of recipe components of a user-centric-based recipe selected by the main control circuit 104. By one approach, the user interface product 102 may compare recipe components of a user-centric-based recipe with a listing of a plurality of food items available in one or more food pantries associated with the user through the user-pantry inventory database 130. In one configuration, the user-pantry inventory database 130 associated with the user may store the listing of the plurality of food items available in the one or more food pantries associated with one or more houses of the user. By one approach, based on the comparison, the user interface product 102 may determine one or more unavailable recipe components of the user-centric-based recipe. Alternatively or in addition to, the user interface product 102 may submit a request for a delivery of the one or more unavailable recipe components. In one example, the user interface product 102 may be configured by the user to automatically submit a request for delivery of recipe components of a user-centric-based recipe that are missing in the user's food pantry. In one configuration, the user interface product 102 may submit the request through a blockchain network to ensure security and authenticity of the delivery request. In another example, the user interface product 102 may submit the request for delivery after receiving an order request input from the user. Alternatively or in addition to, the user interface product 102 may create a meal plan based on user-centric-based recipes received over a period of time. For example, based on data stored in the main database 106, the personal database 108, the taste preference attribute database 110, the recipe preference database 112, the value database 114, the user profile 116, and/or user-centric-based recipes provided and/or selected over a period of time, the main control circuit 104 may create a meal plan particular to the user. By one example, the meal plan may include suggested dishes along with corresponding recipes for a day, a week, a month, and/or a year. In one configuration, the main control circuit 104 and/or the user interface product 102 may suggest a meal plan based on the odor smelled by the user and/or selected recipes that are based on the odor smelled by the user over a period of time. In another example, in creating a meal plan, the main control circuit 104 may determine which one or more particular recipes that have been indicated by the user over a period of time as being only a measure of like. In response to the determination, the main control circuit 104 may provide the one or more particular recipes to the user interface product 102 to include the one or more particular recipes in a meal plan created particularly for the user.

In some embodiments, alternatively or in addition to determining that a filter parameter is not associated with a location coordinate, the user interface product 102 may prompt the user to provide one or more food categories associated with an odor. For example, a user may have smelled an odor emanating from a food truck selling Korean food at an event. Wondering what the odor is, the user may trigger the user interface product 102 to determine what the odor is. In an scenario where the user interface product 102 is unable to determine a filter parameter associated with a location coordinate of the portable user electronic device 118 of the user, the user interface product 102 may prompt the user to provide one or more food categories associated with an odor smelled by the user. In such a scenario, the food truck may not have been associated with a fixed location and, thus, the stored satellite mappings accessible to the user interface product 102 and/or the main control circuit 104 may not have information and/or data corresponding to a location coordinate that is within a threshold distance from a food truck that sells Korean food. In response, the user interface product 102 may prompt the user to provide one or more food categories associated with the odor. For example, the user may provide an input corresponding to Korean food to the user interface product 102. In one example, the user interface product 102 may have a listing of various categories (e.g., food groups, food classifications, dish types, recipe categories, cuisines, or the like) that a user may select. In another example, the user may provide an input typing in one or more words that the user may regard as describing the odor the user smelled. By one approach, the food categories provided by the user may be received by the main control circuit 104. In one configuration, based on the food categories, the main control circuit 104 may determine a second filtered set of recipe components from the first set of associations of the plurality of recipe components stored in the main database 106. For example, the main control circuit 104 may filter out, from a list used to determine recipe components of the odor, all set of chemicals not associated with the Korean food. Thus, the determination of the recipe components of the odor using the second filtered set of recipe components may be more efficient and/or faster than comparing the detected chemicals in the odor with all set of chemicals stored in the main database 106. By one approach, using a filtered set of recipe components determined by the food category enables a more efficient and/or a faster searching and/or comparing of the detected chemicals in the odor and provides a more efficient and/or a faster determination of a recipe component matching one of the detected chemicals of the odor sensor 120. As such, the main control circuit 104 may determine a second recipe component from the second filtered set of recipe components that has a chemical makeup including one or more of the chemicals detected by the odor sensor 120. Similarly, based on the one or more food categories, the main control circuit 104 may efficiently and/or quickly determine a second filtered set of recipes from the second set of associations of the plurality of recipes stored in the main database 106. Continuing the illustrative non-limiting example above, the second filtered set of recipes may only include those recipes associated with Korean food. Thus, one or more uses of filtered parameters associated with a location coordinate and/or food categories enhances the processing of the main control circuit 104 and/or the user interface product 102 by efficiently and/or swiftly providing a user of identification of recipe components of an odor the user is smelling and/or recipes that may be associated with the odor the user is smelling. As such, alternatively or in addition to, the main control circuit 104 may determine one or more second recipes from the second filtered set of recipes that includes the determined second recipe component and provide the one or more second recipes to the user interface product 102 via the internet network 128.

In some embodiments, the user interface product 102 in cooperation with the local memory 126 may store a local set of associations of a plurality of recipe components with a plurality of corresponding set of chemicals of the main database 106. By one approach, the local set may include a set of common recipe components found in a plurality of recipes stored in the main database 106. Alternatively or in addition to, the local set may include another set of common recipe components that were previously detected by the odor sensor 120 based on a first set of odors queried by a plurality of users of a plurality of user interface products communicatively coupled to the main control circuit 104 and a second set of odors queried by the plurality of users with at least one matching attribute that is within a threshold value of a corresponding attribute of a user associated with the portable user electronic device 118. In one configuration, in response to determining the second recipe components from the local set of associations, the user interface product 102 may cause a display device associated with the portable user electronic device 118 to show a listing of the determined second recipe components. As such, the second recipe components displayed on a display device is a result of a determination of recipe components from a set of locally stored associations instead of a filtered set of associations from a database remote from the user interface product 102 and/or the portable user electronic device 118. Alternatively or in addition to, the user interface product 102 may cause the local memory 126 and the personal database 108 to store the second recipe components that are subsequently shown to the display device and associated with the user profile 116 of a user of the portable user electronic device 118. Thus, by using a set of locally stored associations instead of a filtered set of associations from a database remote from the user interface product 102 and/or the portable user electronic device 118 to determine recipe components associated with an odor enhances the processing of the main control circuit 104 and/or the user interface product 102 by efficiently and/or swiftly providing a user of identification of recipe components of an odor the user is smelling and/or recipes that may be associated with the odor the user is smelling. In some embodiments, the systems and/or the methods described herein may be applicable to a home hub device integrated with and/or cooperatively working with an odor sensor. In some embodiments, the main control circuit 104 and/or the user interface product 102 may suggest a different recipe component that may enhance the determined recipe components of an odor. For example, the main control circuit 104 and/or the user interface product 102 may determine that the different recipe component is usually combined with and/or accompanied with the determined recipe components on one or more recipes. In yet some embodiments, the main control circuit 104 and/or the user interface product 102 may broadcast to one or more other portable user electronic devices the determined recipe components of an odor to enable the main control circuit 104 to suggest one or more recipes to one or more users associated with the one or more other portable user electronic devices.

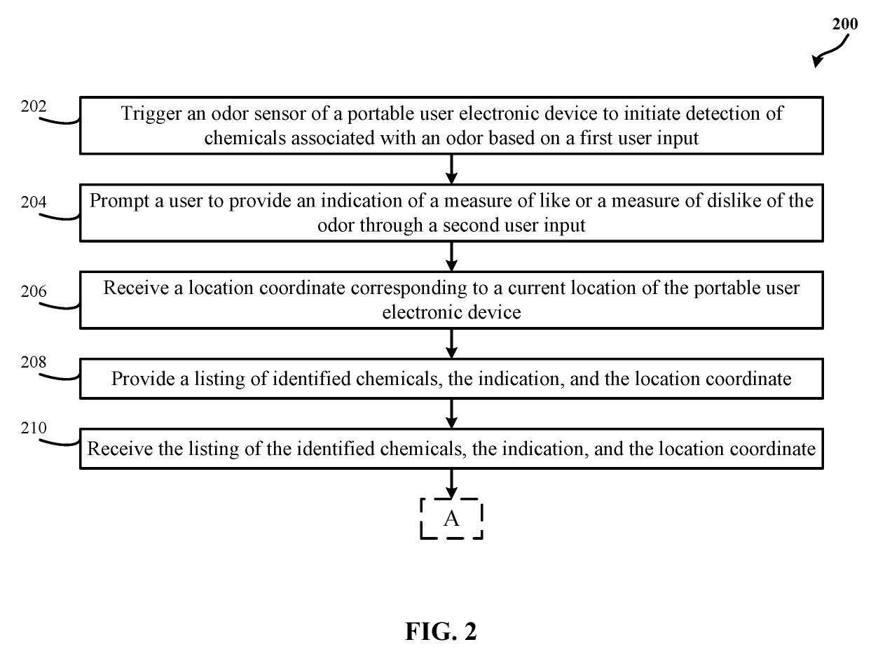

FIG. 2 shows a flow diagram of an exemplary method 200 for recommending items for purchase based on smell of food in accordance with some embodiments. By one approach, the exemplary method 200 may be implemented in the system 100 of FIG. 1. By another approach, one or more steps of the method 200 may be implemented in and/or by the main control circuit 104, the user interface product 102, and/or the portable user electronic device control circuit 124 of FIG. 1. The method 200 includes, at step 202, triggering an odor sensor of a portable user electronic device to initiate detection of chemicals associated with an odor based on a first user input through the portable user electronic device. By one approach, the method 200 may include, at step 204, prompting a user to provide an indication of a measure of like or a measure of dislike of the odor through a second user input. In one example, a user interface product may prompt the user via the portable user electronic device. In another example, the second user input may be provided via the portable user electronic device. By another approach, the method 200 may include, at step 206, receiving a location coordinate corresponding to a current location of the portable user electronic device. By another approach, the method 200 may include, at step 208, providing a listing of identified chemicals, the indication, and/or the location coordinate via an internet network through a transceiver of the portable user electronic device. In one configuration, the identified chemicals may be detected by the odor sensor and identified by a portable user electronic device control circuit of the portable user electronic device. By yet another approach, the method 200 may include, at step 210, receiving the listing of the identified chemicals, the indication, and/or the location coordinate. In one example, a main control circuit may receive the listing of the identified chemicals, the indication, and/or the location coordinate. In one configuration, the main control circuit may be communicatively coupled to a main database, the portable user electronic device control circuit, and/or the user interface product via the internet network. In another configuration, the main database may store a first set of associations of a plurality of recipe components with a plurality of corresponding set of chemicals and a second set of associations of a plurality of recipes with a plurality of corresponding set of recipe components.

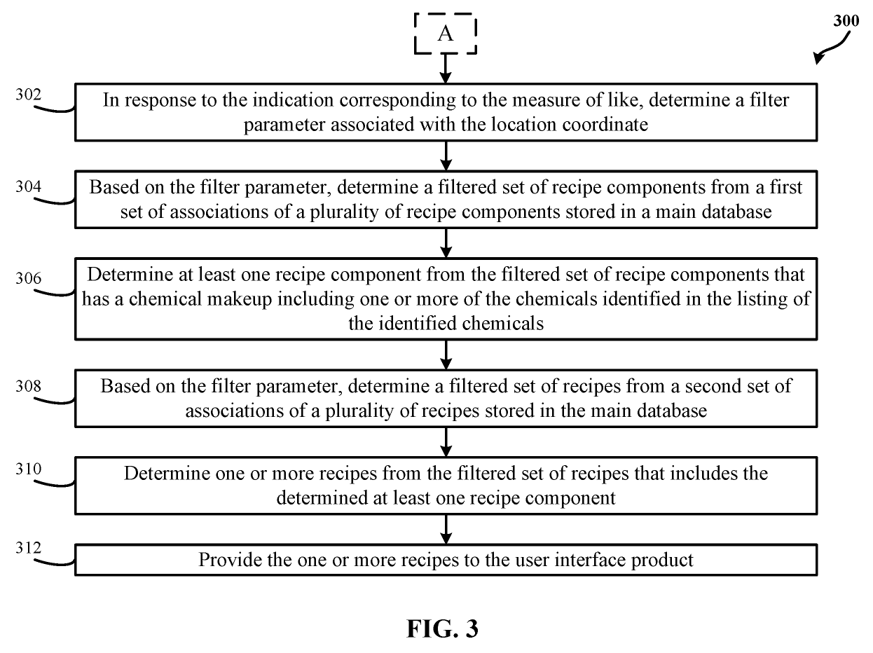

FIG. 3 shows a flow diagram of an exemplary method 300 of recommending items for purchase based on smell of food in accordance with some embodiments. By one approach, the exemplary method 300 may be implemented in the system 100 of FIG. 1. By another approach, the method 300 may be implemented in the main control circuit 104 of FIG. 1. By another approach, one or more steps of the method 300 may be implemented in and/or by the main control circuit 104, the user interface product 102, and/or the portable user electronic device control circuit 124 of FIG. 1. By yet another approach, the method 300 and/or one or more steps of the method may be included in and/or performed in cooperation with the method 200 of FIG. 2. The method 300 may include, at step 302, determining a filter parameter associated with a location coordinate in response to an indication corresponding to a measure of like. In one configuration, the method 300 may include, at step 304, determining, based on the filter parameter, a filtered set of recipe components from a first set of associations of a plurality of recipe components that may be stored in the main database. In another configuration, the method 300 may include, at step 306, determining at least one recipe component from the filtered set of recipe components that has a chemical makeup including one or more of chemicals identified in the listing of identified chemicals. In another configuration, the method 300 may include, at step 308, determining, based on the filter parameter, a filtered set of recipes from a second set of associations of the plurality of recipes stored in the main database. In another configuration, the method 300 may include, at step 310, determining one or more recipes from the filtered set of recipes that includes the determined at least one recipe component. In yet another configuration, the method 300 may include, at step 312, providing the one or more recipes to the user interface product via the internet network.

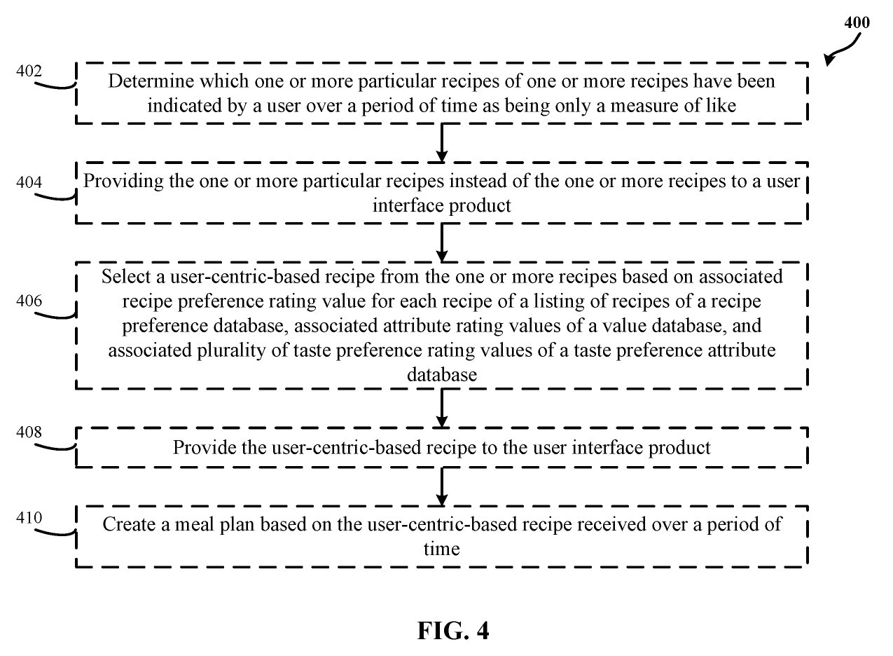

FIG. 4 shows a flow diagram of an exemplary method 400 of recommending items for purchase based on smell of food in accordance with some embodiments. By one approach, the exemplary method 400 may be implemented in the system 100 of FIG. 1. By another approach, one or more steps of the method 400 may be implemented in and/or by the main control circuit 104, the user interface product 102, and/or the portable user electronic device control circuit 124 of FIG. 1. By yet another approach, the method 400 and/or one or more steps of the method may optionally be included in and/or performed in cooperation with the method 200 of FIG. 2 and/or the method 300 of FIG. 3. The method 400 may include, at step 402, determining which one or more particular recipes of one or more recipes have been indicated by a user over a period of time as being only a measure of like. In one configuration, the method 400 may include providing the one or more particular recipes instead of the one or more recipes to a user interface product, at step 404. In another configuration, the method 400 may include, at step 406, selecting a user-centric-based recipe from the one or more recipes based on associated recipe preference rating value for each recipe of a listing of recipes of a recipe preference database, associated attribute rating values of a value database, and/or associated plurality of taste preference rating values of a taste preference attribute database. In one example, the main control circuit may select the user-centric-based recipe. By one approach, the recipe preference database, the value database, and/or the taste preference database may be communicatively coupled to the user interface product, the main control circuit, and/or the portable user electronic device control circuit via an internet network. In another configuration, the method 400 may include, at step 408, providing the user-centric-based recipe to the user interface product via the internet network. In yet another configuration, the method 400 may include creating a meal plan based on the user-centric-based recipe received over a period of time, at step 410.

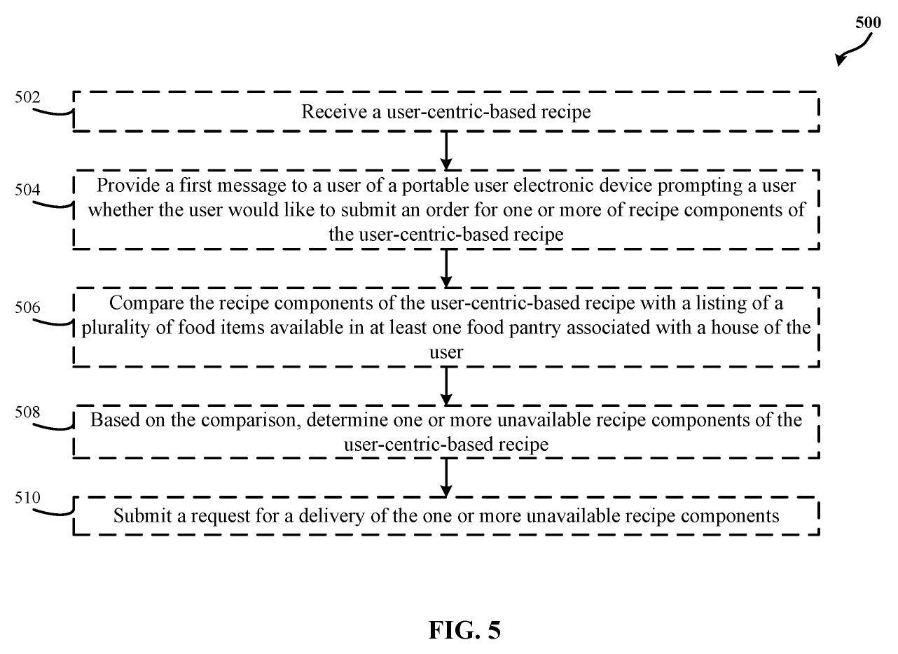

FIG. 5 shows a flow diagram of an exemplary method 500 of recommending items for purchase based on smell of food in accordance with some embodiments. By one approach, the exemplary method 500 may be implemented in the system 100 of FIG. 1. By another approach, one or more steps of the method 500 may be implemented in and/or by the main control circuit 104, the user interface product 102, and/or the portable user electronic device control circuit 124 of FIG. 1. By yet another approach, the method 500 and/or one or more steps of the method may optionally be included in and/or performed in cooperation with the method 200 of FIG. 2, the method 300 of FIG. 3, and/or the method 400 of FIG. 4. The method 500 may include, at step 502, receiving a user-centric-based recipe via the internet network. In one example, a user interface product may receive the user-centric-based recipe. By one approach, the method 500 may include, at step 504, providing a first message to a user of a portable user electronic device prompting a user whether the user would like to submit an order for one or more of recipe components of the user-centric-based recipe. By another approach, the method 500 may include comparing recipe components of the user-centric-based recipe with a listing of a plurality of food items available in at least one food pantry associated with a house of the user, at step 506. In one example, the listing of the plurality of food items available in the at least one food pantry is stored in a user-pantry inventory database. By another approach, the method 500 may include, at step 508, determining, based on the comparison, one or more unavailable recipe components of the user-centric-based recipe. By yet another approach, the method 500 may include, at step 510, submitting a request for a delivery of the one or more unavailable recipe components.

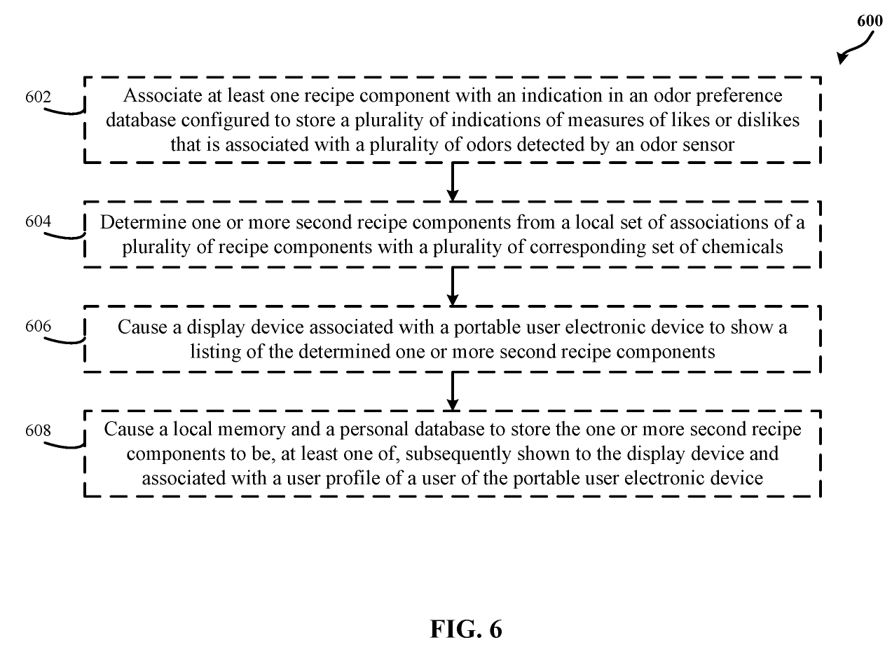

FIG. 6 shows a flow diagram of an exemplary method 600 of recommending items for purchase based on smell of food in accordance with some embodiments. By one approach, the exemplary method 600 may be implemented in the system 100 of FIG. 1. By another approach, one or more steps of the method 600 may be implemented in and/or by the main control circuit 104, the user interface product 102, and/or the portable user electronic device control circuit 124 of FIG. 1. By yet another approach, the method 600 and/or one or more steps of the method may optionally be included in and/or performed in cooperation with the method 200 of FIG. 2, the method 300 of FIG. 3, the method 400 of FIG. 4, and/or the method 500 of FIG. 5. The method 600 may include, at step 602, associating at least one recipe component with an indication in an odor preference database configured to store a plurality of indications of measures of likes or dislikes that is associated with a plurality of odors detected by an odor sensor. By one approach, the method 600 may include, at step 604, determining one or more second recipe components from a local set of associations of a plurality of recipe components with a plurality of corresponding set of chemicals. In one example, the local set may be stored in a local memory associated with a user interface product. In such an example, the local set may comprise a set of common recipe components found in a plurality of recipes. By another approach, the method 600 may include causing a display device associated with a portable user electronic device to show a listing of the determined one or more second recipe components, at step 606. By yet another approach, the method 600 may include, at step 608, causing the local memory and/or a personal database to store the one or more second recipe components to be subsequently shown to the display device and/or associated with a user profile of a user of the portable user electronic device. In one example, the local memory and/or the personal database may be communicatively coupled to a main control circuit.

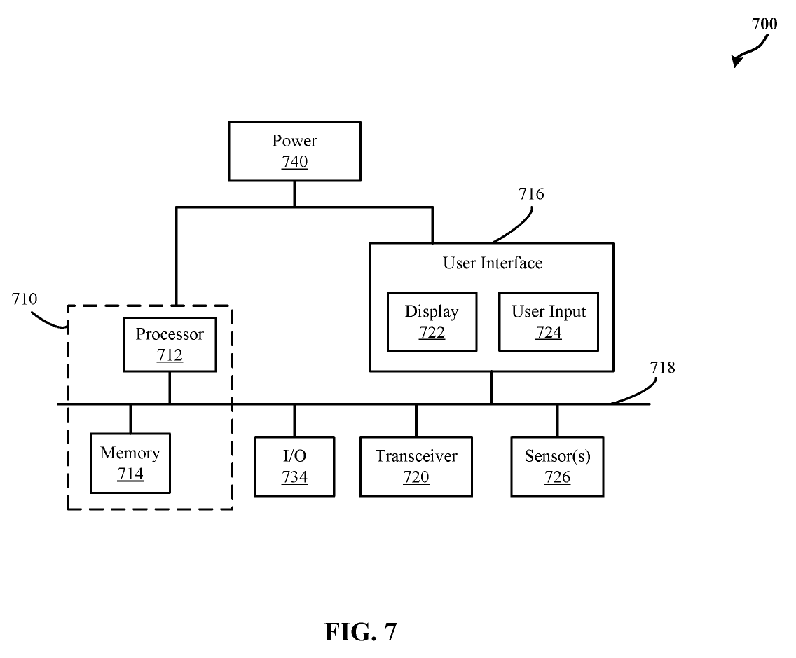

Further, the circuits, circuitry, systems, devices, processes, methods, techniques, functionality, services, servers, sources and the like described herein may be utilized, implemented and/or run on many different types of devices and/or systems. FIG. 7 illustrates an exemplary system 700 that may be used for implementing any of the components, circuits, circuitry, systems, functionality, apparatuses, processes, or devices of the system 100 of FIG. 1, the method 200 of FIG. 2, the method 300 of FIG. 3, the method 400 of FIG. 4, the method 500 of FIG. 5, the method 600 of FIG. 6, and/or other above or below mentioned systems or devices, or parts of such circuits, circuitry, functionality, systems, apparatuses, processes, or devices. For example, the system 700 may be used to implement some or all of the system 100 for recommending items for purchase based on smell of food, the user interface product 102, the main control circuit 104, the main database 106, the personal database 108, the taste preference attribute database 110, the recipe preference database 112, the value database, 114, the user-pantry inventory database 130, the user profile 116, the portable user electronic device 118, the odor sensor 120, the transceiver 122, the portable user electronic device control circuit 124, the local memory 126, and/or other such components, circuitry, functionality and/or devices. However, the use of the system 700 or any portion thereof is certainly not required.

By way of example, the system 700 may comprise a processor module (or a control circuit) 712, memory 714, and one or more communication links, paths, buses or the like 718. Some embodiments may include one or more user interfaces 716, and/or one or more internal and/or external power sources or supplies 740. The control circuit 712 can be implemented through one or more processors, microprocessors, central processing unit, logic, local digital storage, firmware, software, and/or other control hardware and/or software, and may be used to execute or assist in executing the steps of the processes, methods, functionality and techniques described herein, and control various communications, decisions, programs, content, listings, services, interfaces, logging, reporting, etc. Further, in some embodiments, the control circuit 712 can be part of control circuitry and/or a control system 710, which may be implemented through one or more processors with access to one or more memory 714 that can store instructions, code and the like that is implemented by the control circuit and/or processors to implement intended functionality. In some applications, the control circuit and/or memory may be distributed over a communications network (e.g., LAN, WAN, Internet) providing distributed and/or redundant processing and functionality. Again, the system 700 may be used to implement one or more of the above or below, or parts of, components, circuits, systems, processes and the like. For example, the system 700 may implement the system 100 for recommending items for purchase based on smell of food with the portable user electronic device control circuit 124 and/or the main control circuit 104 being the control circuit 712.

The user interface 716 can allow a user to interact with the system 700 and receive information through the system. In some instances, the user interface 716 includes a display 722 and/or one or more user inputs 724, such as buttons, touch screen, track ball, keyboard, mouse, etc., which can be part of or wired or wirelessly coupled with the system 700. Typically, the system 700 further includes one or more communication interfaces, ports, transceivers 720 and the like allowing the system 700 to communicate over a communication bus, a distributed computer and/or communication network (e.g., a local area network (LAN), the Internet, wide area network (WAN), etc.), communication link 718, other networks or communication channels with other devices and/or other such communications or combination of two or more of such communication methods. Further the transceiver 720 can be configured for wired, wireless, optical, fiber optical cable, satellite, or other such communication configurations or combinations of two or more of such communications. Some embodiments include one or more input/output (I/O) interface 734 that allow one or more devices to couple with the system 700. The I/O interface can be substantially any relevant port or combinations of ports, such as but not limited to USB, Ethernet, or other such ports. The I/O interface 734 can be configured to allow wired and/or wireless communication coupling to external components. For example, the I/O interface can provide wired communication and/or wireless communication (e.g., Wi-Fi, Bluetooth, cellular, RF, and/or other such wireless communication), and in some instances may include any known wired and/or wireless interfacing device, circuit and/or connecting device, such as but not limited to one or more transmitters, receivers, transceivers, or combination of two or more of such devices.

In some embodiments, the system may include one or more sensors 726 to provide information to the system and/or sensor information that is communicated to another component, such as the main control circuit 104, the user interface product 102, the portable user electronic device control circuit 124, the odor sensor 120, the portable user electronic device 118, the local memory 126, the main database 106, the personal database 108, the taste preference attribute database 110, the recipe preference database 112, the value database, 114, the user-pantry inventory database 130, the user profile 116, etc. The sensors can include substantially any relevant sensor, such as temperature sensors, distance measurement sensors (e.g., optical units, sound/ultrasound units, etc.), optical based scanning sensors to sense and read optical patterns (e.g., bar codes), radio frequency identification (RFID) tag reader sensors capable of reading RFID tags in proximity to the sensor, and other such sensors. The foregoing examples are intended to be illustrative and are not intended to convey an exhaustive listing of all possible sensors. Instead, it will be understood that these teachings will accommodate sensing any of a wide variety of circumstances in a given application setting.

The system 700 comprises an example of a control and/or processor-based system with the control circuit 712. Again, the control circuit 712 can be implemented through one or more processors, controllers, central processing units, logic, software and the like. Further, in some implementations the control circuit 712 may provide multiprocessor functionality.

The memory 714, which can be accessed by the control circuit 712, typically includes one or more processor readable and/or computer readable media accessed by at least the control circuit 712, and can include volatile and/or nonvolatile media, such as RAM, ROM, EEPROM, flash memory and/or other memory technology. Further, the memory 714 is shown as internal to the control system 710; however, the memory 714 can be internal, external or a combination of internal and external memory. Similarly, some or all of the memory 714 can be internal, external or a combination of internal and external memory of the control circuit 712. The external memory can be substantially any relevant memory such as, but not limited to, solid-state storage devices or drives, hard drive, one or more of universal serial bus (USB) stick or drive, flash memory secure digital (SD) card, other memory cards, and other such memory or combinations of two or more of such memory, and some or all of the memory may be distributed at multiple locations over the computer network. The memory 714 can store code, software, executables, scripts, data, content, lists, programming, programs, log or history data, user information, customer information, product information, and the like. While FIG. 7 illustrates the various components being coupled together via a bus, it is understood that the various components may actually be coupled to the control circuit and/or one or more other components directly.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A system for recommending items for purchase based on smell of food comprising:
a user interface product operable on a portable user electronic device comprising an odor sensor configured to detect chemicals associated with an odor, a transceiver, and a portable user electronic device control circuit configured to identify the chemicals detected by the odor sensor, wherein the user interface product cooperated with the portable user electronic device control circuit is configured to:
trigger the odor sensor to initiate detection of the chemicals associated with the odor based on a first user input through the portable user electronic device;
prompt a user to provide an indication of a measure of like or a measure of dislike of the odor through a second user input via the portable user electronic device;
receive a location coordinate corresponding to a current location of the portable user electronic device; and
provide a listing of the identified chemicals, the indication, and the location coordinate via the transceiver through an internet network;
a main database communicatively coupled to the portable user electronic device control circuit and the user interface product via the internet network, the main database configured to store a first set of associations of a plurality of recipe components with a plurality of corresponding set of chemicals, and a second set of associations of a plurality of recipes with a plurality of corresponding set of recipe components; and
a main control circuit communicatively coupled to the main database, the portable user electronic device control circuit, and the user interface product via the internet network, the main control circuit configured to:
receive the listing of the identified chemicals, the indication, and the location coordinate;
in response to the indication of the measure of like, determine a filter parameter associated with the location coordinate;
based on the filter parameter, determine a filtered set of recipe components from the first set of associations of the plurality of recipe components stored in the main database;
determine at least one recipe component from the filtered set of recipe components that has a chemical makeup including one or more of the chemicals identified in the listing of the identified chemicals;
based on the filter parameter, determine a filtered set of recipes from the second set of associations of the plurality of recipes stored in the main database;
determine one or more recipes from the filtered set of recipes that includes the determined at least one recipe component; and
provide the one or more recipes to the user interface product via the internet network.

2. The system of claim 1, wherein the indication comprises a rating of the odor by the user, and wherein the rating corresponds to at least one of the user's degree of likeness of the odor or the user's degree of dis-likeness of the odor.

3. The system of claim 1, wherein the indication comprises a rating of the odor by the user, and wherein the rating corresponds to at least one of the user's degree of emotional response elicited by the odor.

4. The system of claim 1, wherein the user interface product is further configured to:
receive the one or more recipes via the internet network;
provide a first message to a user of the portable user electronic device prompting the user to select one of the one or more recipes; and
provide a second message to the user prompting the user whether the user would like to submit an order for one or more recipe components of the selected recipe.

5. The system of claim 4, further comprising a plurality of databases comprising a value database storing a plurality of recipe attributes and corresponding number of times each attribute of the plurality of recipe attributes is associated with the selected recipe, wherein the corresponding number of times for each attribute is tallied over a period of time, and wherein the user interface product is further configured to:
provide a third message to the user prompting the user to provide an attribute rating value to each of the plurality of recipe attributes relative to each of the one or more recipes; and
tally each of the plurality of recipe attributes in the value database based on the attribute rating value provided by the user to each of the plurality of recipe attributes of each of the one or more recipes.

6. The system of claim 1, further comprising a plurality of databases comprising a taste preference attribute database, wherein the user interface product is further configured to:
receive the at least one recipe component via the internet network;
provide a first message to a user of the portable user electronic device prompting the user to provide a taste preference rating value to the at least one recipe component; and
associate, in cooperation with the portable user electronic device control circuit, the taste preference rating value with the at least one recipe component in the taste preference attribute database.

7. The system of claim 1, further comprising a plurality of databases comprising a recipe preference database, wherein the user interface product is further configured to:
receive the one or more recipes via the internet network;
provide a first message to a user of the portable user electronic device prompting the user to provide a recipe preference rating value to each of the one or more recipes; and
associate, in cooperation with the portable user electronic device control circuit, the recipe preference rating value with each of the one or more recipes in the recipe preference database.

8. The system of claim 1, further comprising a personal database associated with a user of the portable user electronic device, the personal database comprises:
a recipe preference database configured to store a listing of recipes based on the chemicals detected by the odor sensor over a time period, and associated recipe preference rating value for each recipe of the listing of recipes provided by the user over the time period;
a value database configured to store a plurality of recipe attributes with associated attribute rating values provided by the user over the time period, wherein the associated attribute rating values are updated each time the user provides recipe attribute ratings to each of the one or more recipes; and
a taste preference attribute database configured to store a plurality of determined recipe components with associated plurality of taste preference rating values provided by the user over the time period, wherein the plurality of determined recipe components is based on the chemicals detected by the odor sensor over the time period.

9. The system of claim 8, wherein the main control circuit is further configured to:
select a user-centric-based recipe from the one or more recipes based on the associated recipe preference rating value for each recipe of the listing of recipes of the recipe preference database, the associated attribute rating values of the value database, and the associated plurality of taste preference rating values of the taste preference attribute database; and
provide the user-centric-based recipe to the user interface product via the internet network.

10. The system of claim 9, wherein the user interface product is further configured to receive the user-centric-based recipe via the internet network; and
provide a first message to a user of the portable user electronic device prompting the user whether the user would like to submit an order for one or more of recipe components of the user-centric-based recipe.

11. The system of claim 10, further comprising a user-pantry inventory database configured to store a listing of a plurality of food items available in at least one food pantry associated with a house of the user, wherein the user interface product is further configured to:
compare the recipe components of the user-centric-based recipe with the listing of the plurality of food items available in the at least one food pantry;
based on the comparison, determine one or more unavailable recipe components of the user-centric-based recipe; and
submit a request for a delivery of the one or more unavailable recipe components.

12. The system of claim 10, wherein the user interface product is further configured to create a meal plan based on the user-centric-based recipe received over the period of time.

13. The system of claim 1, wherein, in a determination, by the main control circuit, that the filter parameter is not associated with the location coordinate, the user interface product is further configured to:
prompt the user to provide one or more food categories associated with the odor; and
provide the one or more food categories to the main control circuit;
wherein the main control circuit is further configured to:
based on the one or more food categories, determine a second filtered set of recipe components from the first set of associations of the plurality of recipe components stored in the main database;
determine at least one second recipe component from the second filtered set of recipe components that has a chemical makeup including the one or more of the chemicals identified in the listing of the identified chemicals;
based on the one or more food categories, determine a second filtered set of recipes from the second set of associations of the plurality of recipes stored in the main database;
determine one or more second recipes from the second filtered set of recipes that includes the determined at least one second recipe component; and
provide the one or more second recipes to the user interface product via the internet network.

14. The system of claim 1, wherein the user interface product is further configured to:
determine one or more second recipe components from a local set of associations of the plurality of recipe components with the plurality of corresponding set of chemicals, wherein the local set is stored in a local memory associated with the user interface product, and wherein the local set comprises a set of common recipe components found in the plurality of recipes; and
cause a display device associated with the portable user electronic device to show a listing of the determined one or more second recipe components.

15. The system of claim 14, wherein the local set further comprises another set of common recipe components that were previously detected based on at least one of: a first set of odors queried by a plurality of users of a plurality of user interface products communicatively coupled to the main control circuit and a second set of odors queried by the plurality of users with at least one matching attribute that is within a threshold value of a corresponding attribute of a user associated with the portable user electronic device.

16. The system of claim 14, wherein the user interface product is further configured to cause the local memory and a personal database communicatively coupled to the main control circuit to store the one or more second recipe components to be, at least one of, subsequently shown to the display device and associated with a user profile of a user of the portable user electronic device.

17. A method for recommending items for purchase based on smell of food comprising:
triggering, by a user interface product operable on a portable user electronic device, an odor sensor of the portable user electronic device to initiate detection of chemicals associated with an odor based on a first user input through the portable user electronic device;
prompting, by the user interface product, a user to provide an indication of a measure of like or a measure of dislike of the odor through a second user input via the portable user electronic device;
receiving, by the user interface product, a location coordinate corresponding to a current location of the portable user electronic device;
providing, by the user interface product, a listing of identified chemicals, the indication, and the location coordinate via an internet network through a transceiver of the portable user electronic device, wherein the identified chemicals are detected by the odor sensor and identified by a portable user electronic device control circuit of the portable user electronic device;
receiving, by a main control circuit communicatively coupled to a main database, the portable user electronic device control circuit, and the user interface product via the internet network, the listing of the identified chemicals, the indication, and the location coordinate, wherein the main database is configured to store a first set of associations of a plurality of recipe components with a plurality of corresponding set of chemicals, and a second set of associations of a plurality of recipes with a plurality of corresponding set of recipe components;
in response to the indication corresponding to the measure of like, determining a filter parameter associated with the location coordinate;
based on the filter parameter, determining a filtered set of recipe components from the first set of associations of the plurality of recipe components stored in the main database;
determining, by the main control circuit, at least one recipe component from the filtered set of recipe components that has a chemical makeup including one or more of the chemicals identified in the listing of the identified chemicals;
based on the filter parameter, determining, by the main control circuit, a filtered set of recipes from the second set of associations of the plurality of recipes stored in the main database;
determining, by the main control circuit, one or more recipes from the filtered set of recipes that includes the determined at least one recipe component; and
providing, by the main control circuit, the one or more recipes to the user interface product via the internet network.

18. The method of claim 17, wherein the indication comprises a rating of the odor by the user, and wherein the rating corresponds to at least one of: the user's degree of likeness of the odor, the user's degree of dis-likeness of the odor, and at least one of the user's degree of emotional response elicited by the odor.

19. The method of claim 17, further comprising:
determining which one or more particular recipes of the one or more recipes have been indicated by the user over a period of time as being only the measure of like; and
providing the one or more particular recipes instead of the one or more recipes to the user interface product.

20. The method of claim 17, further comprising:
selecting, by the main control circuit, a user-centric-based recipe from the one or more recipes based on associated recipe preference rating value for each recipe of a listing of recipes of a recipe preference database, associated attribute rating values of a value database, and associated plurality of taste preference rating values of a taste preference attribute database, wherein the recipe preference database, the value database, and the taste preference database are communicatively coupled to the user interface product, the main control circuit, and the portable user electronic device control circuit via the internet network;
providing, by the main control circuit, the user-centric-based recipe to the user interface product via the internet network; and
creating, by the user interface product, a meal plan based on the user-centric-based recipe received over a period of time.

21. The method of claim 20, further comprising:
receiving, by the user interface product, the user-centric-based recipe via the internet network; and
providing, by the user interface product, a first message to a user of the portable user electronic device prompting the user whether the user would like to submit an order for one or more of recipe components of the user-centric-based recipe.

22. The method of claim 21, further comprising:
comparing, by the user interface product, the recipe components of the user-centric-based recipe with a listing of a plurality of food items available in at least one food pantry associated with a house of the user, wherein the listing of the plurality of food items available in the at least one food pantry is stored in a user-pantry inventory database;
based on the comparing, determining, by the user interface product, one or more unavailable recipe components of the user-centric-based recipe; and
submitting, by the user interface product, a request for a delivery of the one or more unavailable recipe components.

23. The method of claim 17, further comprising associating the at least one recipe component with the indication in an odor preference database configured to store a plurality of indications of measures of likes or dislikes that is associated with a plurality of odors detected by the odor sensor.

24. The method of claim 17 further comprising:
determining, by the user interface product, one or more second recipe components from a local set of associations of the plurality of recipe components with the plurality of corresponding set of chemicals, wherein the local set is stored in a local memory associated with the user interface product, and wherein the local set comprises a set of common recipe components found in the plurality of recipes; and
causing, by the user interface product, a display device associated with the portable user electronic device to show a listing of the determined one or more second recipe components.

25. The method of claim 24, wherein the local set further comprises another set of common recipe components that were previously detected based on at least one of: a first set of odors queried by a plurality of users of a plurality of user interface products communicatively coupled to the main control circuit and a second set of odors queried by the plurality of users with at least one matching attribute that is within a threshold value of a corresponding attribute of a user associated with the portable user electronic device.

26. The method of claim 24, further comprising causing, by the user interface product, the local memory and a personal database communicatively coupled to the main control circuit to store the one or more second recipe components to be, at least one of, subsequently shown to the display device and associated with a user profile of a user of the portable user electronic device.
